(19) United States
(12) Patent Application Publication
(10) Pub. No.: US 2017/0266101 A1
(43) Pub. Date: Sep. 21, 2017

(54) KERATINOUS FIBER CARE PRODUCT AND METHOD

(71) Applicant: Henkel AG & Co. KGaA, Duesseldorf (DE)

(72) Inventors: Thomas Foerster, Duesseldorf (DE); Rolf Bayersdoerfer, Hamburg (DE); Thorsten Knappe, Schenefeld (DE)

(73) Assignee: Henkel AG & Co. KGaA, Duesseldorf (DE)

(21) Appl. No.: 15/532,123

(22) PCT Filed: Nov. 2, 2015
(86) PCT No.: PCT/EP2015/075392

§ 371 Date: Jun. 1, 2017

(30) Foreign Application Priority Data
Dec. 10, 2014 (DE) 10 2014 225 436.1

(51) Int. Cl. A61K 8/87; A61K 8/81; B05B 1/24; A61Q 5/06; A45D 34/00; A61K 8/34; A61K 8/64

(52) U.S. Cl. CPC A61K 8/87; A61K 8/34; A61K 8/8176; A61K 8/8182; A61K 8/64; A61Q 5/06; A45D 34/00; B05B 1/24; A61K 2800/87; A45D 2200/155

(57) ABSTRACT

Disclosed is a cosmetic product comprising a) a cosmetic preparation containing, in relation to the total weight of the preparation, a1) at least about 88 wt % polar solvent; a2) at least about 0.1 wt % film-forming polymer; b) a device for flash evaporating cosmetic preparation a), the percentage by weight of components a1) and a2) being more than about 92% of the total weight of cosmetic preparation a). Also disclosed is a method using corresponding products and the use of cosmetic preparation a) as a process material in a flash evaporation device.

### CROSS-REFERENCE TO RELATED APPLICATION

This application is a U.S. National-Stage entry under 35 U.S.C §371 based on International Application No. PCT/EP2015/075392 filed Nov. 2, 2015 which was published under Article 21(2) and which claims priority to German Application No. 102014225436.1, filed Dec. 10, 2014, which are all hereby incorporated in their entirety by reference.

### TECHNICAL FIELD

The present patent application relates to the technical field of care of keratin-containing fibers, in particular human hair. The subject matter of the application is thus specific cosmetic hair formulations, which are suitable for application to keratin-containing fibers by employing a flash vaporization process. In addition, the subject matter of the present disclosure is the use of these cosmetic hair formulations in devices for flash vaporization and methods for temporary shaping of keratin-containing fibers.

### BACKGROUND

Having an attractive hairstyle today is generally considered an essential part of a well-groomed appearance. Today's chic hairstyles based on current fashion trends can be maintained only by using hair shaping methods.

A number of different techniques are used for permanent or temporary shaping of human hair, including relaxation, straightening, backcombing or hair-setting. These techniques are based on the use of cosmetic agents that have a hair-setting effect, so-called styling agents, such as hair sprays, hair waxes, hair gels, hair-setting products, permanent waves, styling sprays.

Alternatively or in addition to treatment with hair-setting cosmetic agents, hair can also be subjected to a thermal treatment.

To create waves or curls in straight hair, the hair is shaped by application of heat with the help of a hair dryer, for example, using a diffuser or a curling iron. Straight hair is wrapped around the heated curling iron or the diffuser, during which temperatures up to 250° C. may be reached. In this method, styling sprays, which are applied to the hair before the actual thermal treatment, are usually used to improve the styling results.

Special straightening irons are used for straightening hair. Straightening irons have two parallel metal or ceramic plates, through which the hair is pulled after the plates are hot. Commercial straightening irons can be heated to temperatures in the range of 150° C. to 250° C. The goal of the using such a straightening iron is to straighten curly hair or straightened hair by thermal/physical methods. If hair is to be straightened by using a straightening iron, then a styling spray of the type also known as a straightening iron spray is usually applied to the hair in advance as a styling agent for straightening irons. The spray supports the sliding of the iron and the straightening of the hair.

To protect hair from the effects of the high temperatures used for styling hair, heat protectants may be used. When using these heat protectants, the spray application of corresponding cosmetic preparations in particular is extremely important, the preparations usually being applied as pump sprays or aerosol sprays. The cosmetic preparations are therefore fabricated in a dispensing device, from which they are sprayed either by employing a mechanical force effect or with the help of a blowing agent through a valve. Both methods have obvious disadvantages. Pump sprays are not usually suitable for a long-lasting uniform spray application of cosmetic hair preparations, but aerosol sprays are based on the use of blowing agents or propellant gases which do not manifest any cosmetic effect, on the one hand, and may involve a risk to the consumer if handled inappropriately, on the other hand.

Against this background, there is a demand for alternative methods of atomization of cosmetic hair preparations. Flash vaporization has proven to be one such alternative spray method. Flash vaporization is a method described in the International Patent Application WO 2001/83071 A1 (Henkel), in which a liquid or pasty composition containing a solvent is heated in a sealed space to a temperature higher than the boiling point of the solvent, which thus creates an excess pressure in the composition. When the pressure is releases (throttled), the liquid evaporates and can then be atomized through a suitable nozzle, for example.

Although flash vaporization is basically suitable for spray application of cosmetic hair preparations, at the same time not all cosmetic hair preparations can be atomized by employing a flash vaporization method. On one hand, this is due to the heating of the cosmetic preparation, which is necessary for flash vaporization, but on the other hand, is due to the specific properties of the spray mist formed by flash vaporization, for example, the size of the droplets produced and the density of droplets in the spray mist.

### BRIEF SUMMARY

A cosmetic product is provided herein. The cosmetic product includes a) a cosmetic preparation including, based on its total weight, a1) at least about 88% by weight polar solvent; and a2) at least about 0.1% by weight film-forming polymer. The cosmetic product further includes b) a device for flash vaporization of the cosmetic preparation a). The amount by weight of components a1) and a2) in the total weight of the cosmetic preparation a) amounts to more than about 92%.

A cosmetic preparation a) is also provided herein. The cosmetic preparation a) includes, based on its total weight, a1) at least about 88% by weight polar solvent; and a2) at least about 0.1% by weight film-forming polymer. The cosmetic product further includes b) a device for flash vaporization of the cosmetic preparation a). The amount by weight of components a1) and a2) in the total weight of the cosmetic preparation a) amounts to more than about 92%. The cosmetic preparation a) is utilized as a material in a device for flash vaporization.

A method for temporary shaping of keratin-containing fibers is also provided herein. The method includes treating the keratin-containing fibers by employing a device for flash vaporization using a cosmetic preparation a). The cosmetic preparation a) includes, based on its total weight, a1) at least about 88% by weight polar solvent; and a2) at least about 0.1% by weight film-forming polymer. The cosmetic product further includes b) a device for flash vaporization of the cosmetic preparation a). The amount by weight of components a1) and a2) in the total weight of the cosmetic preparation a) amounts to more than about 92%.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to make available specific cosmetic hair preparations for temporary shaping of keratin-containing fibers, such that these preparations will be suitable for targeted spray application by employing a device for flash vaporization because of the chemical and physical properties of these preparations. In addition, these preparations should be suitable for achieving an excellent care effect after being applied by employing a flash vaporization process. It has been found that to achieve this object, preparations based on film-forming polymers with a high solvent content are suitable in particular from the variety of known polymer preparations that have a cosmetic effect on hair.

The first subject matter of the present disclosure is thus a cosmetic product, comprising

a) a cosmetic preparation containing based on its total weight
a1) at least about 88% by weight polar solvent;
a2) at least about 0.1% by weight film-forming polymer;
b) a device for flash vaporization of the cosmetic preparation a)
wherein the amount by weight of components a) and b) relative to the total weight of the cosmetic preparation a) amounts to more than about 92%.

The cosmetic preparation a) is liquid. The cosmetic preparation a) may be present in the form of a solution or dispersion, for example, as an emulsion or suspension. Preferred cosmetic preparations a) are in the form of solution or a suspension.

The cosmetic preparation as contemplated herein contains as the first essential ingredient at least about 88% by weight of a polar solvent a). Preferred cosmetic products are characterized in that the amount by weight of the polar solvent a1) in the total weight of the cosmetic preparation a) amounts to at least about 80% by weight, preferably at least about 85% by weight, especially preferably at least about 90% by weight and in particular at least about 95% by weight. Corresponding agents are characterized by a good cosmetic effect with good applicability at the same time.

To improve the application properties of the cosmetic preparations as contemplated herein, while at the same time minimizing the thermal load of any active ingredients or additives in the course of the flash vaporization method, it has proven advantageous to use polar solvents a1) which have a boiling point (20° C., 1013 mbar) between about 50 and about 110° C., preferably between about 70 and about 105° C. Ethanol and water have proven to be particularly suitable and for this reason are the preferred polar solvents a1).

Especially preferred polar solvents a1) or solvent systems include:

polar solvents a1) comprising more than about 80% by weight, preferably more than 85% by weight and in particular more than about 90% by weight water, each based on the total weight of the polar solvent;

polar solvents a1) comprising water and ethanol, wherein the weight ratio of water to ethanol is about 50:1 to about 1:1, preferably about 40:1 to about 5:1 and in particular about 25:1 to about 10:1.

A second essential component of the cosmetic preparations as contemplated herein is the film-forming polymer a2). Both permanently and temporarily cationic, anionic, nonionic and amphoteric polymers are suitable as the film-forming polymers a2). These film-forming polymers may be of a synthetic or natural origin. With respect to the manufacturability, applicability and cosmetic effect of the cosmetic compositions as contemplated herein, it has proven advantageous if the amount by weight of the film-forming polymer in the total weight of the cosmetic preparation amounts to about 0.1% to about 10% by weight, preferably about 0.2% to about 8.0% by weight and in particular about 0.4% to about 5.0% by weight.

Examples of conventional film-forming polymers a2) [PFW1] include acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C₁₋₂ succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol acrylates/acrylamide copolymer, aminoethylpropanediol-AMPD-acrylates/diacetoneacrylamide copolymer, ammonium VA/acrylates copolymer, AMPD-acrylates/diacetoneacrylamide copolymer, AMP-acrylates/allyl methacrylate copolymer, AMP-acrylates/C₁₋₁₈ alkyl acrylates/C₁₋₈ alkyl acrylamide copolymer, AMP-acrylates/diacetoneacrylamide copolymer, AMP-acrylates/dimethylaminoethyl methacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, cornstarch/acrylamide/sodium acrylate copolymer, diethylene glycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MA copolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, poly-beta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, polysilicone-9, polyurethane-1, polyurethane-6, polyurethane-10, polyvinyl acetate, polyvinyl butyral, polyvinyl caprolactam, polyvinyl formamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVP/VA/ltaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethyl siloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

Based on their cosmetic effect, the nonionic polymers, preferably polymers from the group of polyvinylpyrrolidones and vinylpyrrolidone/vinyl acetate copolymers, in particular vinylpyrrolidone/vinyl acetate copolymers form a first group of particularly preferred film-forming polymers a2).

Preferred polyvinylpyrrolidones (INCI designation PVP) are distributed under the brand name Luviskol® (BASF), for example.

The vinylpyrrolidone/vinyl acetate copolymers can be attributed to the monomers i) vinylpyrrolidone and ii) vinyl acetate plus optionally other monomers. Preferred vinylpyrrolidone/vinyl acetate copolymers consist of at least about 90% by weight, preferably at least about 95% by weight and in particular at least about 97% by weight of the monomers i) vinylpyrrolidone and ii) vinyl acetate. Particularly preferred copolymers have been obtained exclusively from the monomers i) vinylpyrrolidone and ii) vinyl acetate.

In the vinylpyrrolidone-vinyl acetate copolymers, vinylpyrrolidone and vinyl acetate may be present in different ratios. Vinylpyrrolidone-vinyl acetate copolymers with a higher vinylpyrrolidone content are water soluble above a vinylpyrrolidone content of about 60%, for example. However, vinylpyrrolidone-vinyl acetate copolymers with a lower vinylpyrrolidone content may be dissolved in ethanol. As contemplated herein, it is preferable if the vinylpyrrolidone-vinyl acetate copolymer a2) has a weight ratio of vinylpyrrolidone to vinyl acetate of about 30:70 to about 70:30.

For example, the vinylpyrrolidone/vinyl acetate copolymers are distributed under the brand names Luviskol® VA 37, Luviskol® VA 55, Luviskol® VA 64 and Luviskol® VA 73 (INCI designation: VP/VA copolymer; CAS number 25086-89-9), each from the company BASF SE.

A second group of film-forming polymers a2) with especially advantageous cosmetic properties is formed by the polyurethanes. The term “polyurethane polymers” is understood to refer to polymers in which at least two monomers, preferably at least three monomers are linked via a urethane group —NH—CO—O—. As contemplated herein, the disclosure also includes such polyurethane polymers which have repeating units —NH—C(O)—NH— that contain urea groups due to the production process, such as those formed by the reaction of isocyanate-terminated prepolymers with compounds containing amino groups. Especially preferred as contemplated herein are polyurethane polymers in which all the monomers are linked via urethane groups and/or groups containing urea groups. Such polyurethane polymers can be obtained by reaction of divalent or polyvalent alcohols with diisocyanates, for example.

Polyurethanes from the group of anionic polyurethane polymers, comprising at least one structural unit of formula (I) and at least one structural unit of formula (II) and at least one structural unit of formula (III) and at least one structural unit of formula (IV) and at least one structural unit of formula (V) are especially preferred:

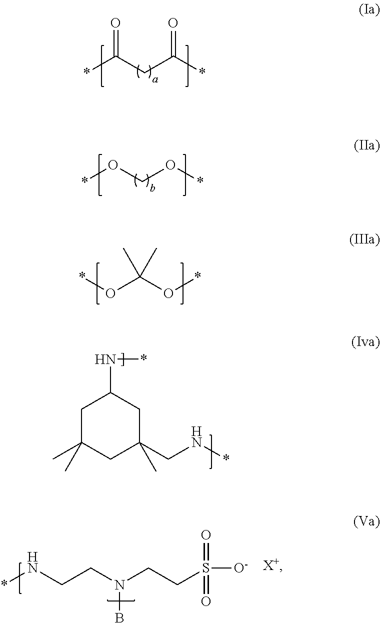

where

a and b, independently of one another, stand for integers from 1 to 20,

R¹, R², R³, R⁴ and R⁵, each independently of one another, stand for a hydrogen atom or a C₁-C₄ alkyl group,

B stands for a hydrogen atom or a direct bond to another structural unit,

X⁺ stands for a physiologically tolerable cation.

According to the above formulas and all the following formulas, a chemical bond characterized by the symbol “*” stands for a free valence of the corresponding structural fragment. The term “free valence” as used here is understood to refer to the number of atomic bonds emanating from the corresponding structural fragment in the position marked with the symbol “*”. Within the scope of the present disclosure, preferably one atomic bond emanates from the positions of the structural fragments marked with the symbol “*” to other structural fragments.

In addition, the term “anionic polyurethane polymers,” as used according to the present disclosure, is understood to include polyurethane polymers, which have at least one structural unit with permanent anionic groups in a protic solvent under standard conditions, wherein the anionic groups must be compensated by counterions while maintaining electroneutrality. Anionic groups are understood as contemplated herein to include in particular carboxyl groups and sulfonic acid groups.

The particularly preferred polyurethane polymers are based on structural units of formula (I) through (V). The R¹ to R⁵ radicals must stand for C₁ to C₄ alkyl groups in the structural units of formulas (III) and (IV). Examples of these include methyl, ethyl, propyl, isopropyl, butyl, sec-butyl, isobutyl and tert-butyl groups.

Anionic polyurethane polymers can be obtained by reacting a prepolymer (V1) that contains isocyanate groups with at least one compound (V2) containing amino groups.

Prepolymers (V1) containing isocyanate groups are used for this purpose; these prepolymers have terminal isocyanate groups, i.e., such polymers (V1) have isocyanate groups on the chain termini, preferably on all chain termini of the polymer. In addition, it is preferable to use prepolymers containing isocyanate groups that are obtained by reacting isocyanates and/or polyisocyanates with one or more polyols, selected from the group including polyether polyols, polycarbonate polyols, polyether polycarbonate polyols and/or polyester polyols. Suitable prepolymers (V1) are therefore in particular the aliphatic, aromatic or cycloaliphatic polyisocyanates that have an NCO functionality of ≧2 and are known per se to those skilled in the art and are therefore suitable in particular.

Especially preferred isocyanates include hexamethylene diisocyanate, isophorone diisocyanate or the isomeric bis-(4,4′-isocyanatocyclohexyl)methanes as well as mixtures of the aforementioned diisocyanates. Especially preferred aliphatic polyester polyols include polyester polyols based on aliphatic carboxylic acids and aliphatic polyols, in particular those based on adipic acid and aliphatic alcohols such as hexane diol and/or neopentyl glycol with an average molecular weight mol. wt. of about 600 to about 3000 g/mol. In addition, polycarbonates, in particular polycarbonate diols having at least one hydroxyl group and an average molecular weight mol. wt. of about 600 to about 3000 g/mol are preferably used; these can be obtained by reaction of carbonic acid derivatives such as diphenyl carbonate, dimethyl carbonate or phosgene with polyols, preferably diols, for example. Polyether polyols such as polytetramethylene glycol polyether and polycarbonate polyols and/or mixtures of these may also preferably be used. The average molecular weight (mol. wt.) can be determined, for example, by employing gel permeation chromatography (GPC) with polystyrene as the internal standard, as described in the Unexamined German Patent Application DE 1999614603.

The compounds (V2) containing amino groups that are used to prepare the anionic polyurethane polymers preferred for use according to the present disclosure are preferably selected from primary and/or secondary amines and/or diamines. In particular the compounds (V2) containing amino groups comprise at least one diamine. The compounds (V2) containing amino groups are preferably selected from compounds containing amino groups and having ionic or ionogenic (ion-forming) groups and compounds containing amino groups, which do not have any ionic or ionogenic groups. Suitable compounds (V2) containing amino groups, which do not contain any ionic or ionogenic groups, are preferably selected from 1,2-ethyenediamine, bis(4-aminocyclohexyl)methane, 1,4-diaminobutane, isophoronediamine, ethanolamine, diethanolamine and diethylenetriamine. Compounds (V2), which contain amino groups and also contain ionic and/or ionogenic groups, preferably have sulfonate groups and/or sulfonic acid groups, especially preferably sodium sulfonate groups. Compounds (V2) containing amino groups and having ionic or ionogenic groups are preferably selected from the salts of 2-(2-aminoethylamino)ethanesulfonic acid, ethylenediaminepropylsulfonic acid or -butylsulfonic acid, 1,2- or 1,3-propylenediamine-β-ethylsulfonic acid as well as taurine.

According to a first specific embodiment of the present disclosure, “a” in the structural unit of formula (I) stands for integers from 1 to 10, preferably from 2 to 8, in particular from 2 to 6, and “b” in the structural unit of formula (II) stands for integers from 1 to 12, preferably from 2 to 10, in particular from 4 to 8.

Within the scope of the preset disclosure, it may also be provided that the radicals R¹, R², R³, R⁴ and R⁵, each independently of one another, in the structural units of formulas (III) and (IV) stand for hydrogen or for a methyl group, in particular for a methyl group.

It is preferable as contemplated herein if X⁺ in the structural unit of formula (I) stands for metal cations of physiologically tolerable metals from the groups Ia, Ib, IIa, IIb, IIIb, VIa or VIII of the periodic system of elements, ammonium ions as well as cationic organic compounds with quaternized nitrogen atoms. Cationic organic compounds can be obtained by protonation of primary, secondary or tertiary organic amines with an acid or by permanent quaternization of said organic amines. Examples of cationic organic ammonium compounds that are suitable within the scope of the present disclosure include 2-ammonioethanol and 2-trimethylammonioethanol.

According to a particularly preferred specific embodiment of the present disclosure, the cosmetic preparation a) as contemplated herein contains at least one anionic polyurethane polymer a), comprising at least one structural unit of formula (Ia) and at least one structural unit of formula (IIa) and at least one structural unit of formula (IIIa) and at least one structure unit of formula (IVa) and at least one structural unit of formula (Va):

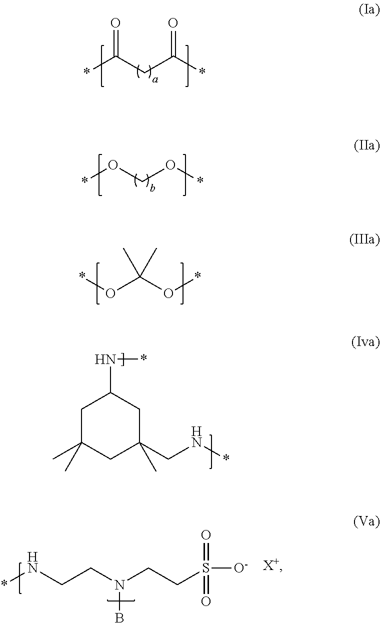

where
a stands for integers from 2 to 6,
b stands for integers from 4 to 8, and
X⁺ stands for a physiologically tolerable cation, in particular sodium. Use of the aforementioned special anionic polyurethane polymers in the cosmetic agent as contemplated herein leads to an excellent hold for a hairstyle, in particular an excellent smoothing effect. In addition, the excellent hairstyle hold is ensured to last for a long time.

A most especially preferred anionic polyurethane polymer within the scope of this embodiment is the polymer known by the INCI designation polyurethane-48. This polymer contains adipic acid (a in the structural unit of formula (Ia) stands for the integer 4) and 1,6-hexanediol (b in the structural unit of formula (IIa) stands for the integer 6).

Anionic polyurethane polymers preferred for use as contemplated herein have a certain glass transition temperature T_{g}. It is therefore preferable within the scope of the present disclosure if the at least one anionic polyurethane polymer a) has a glass transition temperature T_{g} of about −70° C. to about −20° C., preferably of about −65° C. to about −25° C., preferably of about −60° C. to about −30° C., in particular of about −50° C. to about −40° C. The glass transition temperatures determined by employing dynamic differential calorimetry (DSC, differential scanning calorimetry) at a heating rate of about 10K/min, at a starting temperature of at least about 30° C. below the glass transition temperature and a final temperature at least about 30° C. higher than the glass transition temperature. The glass transition temperature T_{g} is obtained as the midpoint temperature according to the tangent method.

The cosmetic products as contemplated herein also comprise, in addition to the cosmetic preparation a), a device for flash vaporization. The term “flash vaporization” within the scope of the present patent application refers to the formation of vapor when there is a drop in pressure in a closed space which is filled with liquid and is under pressure (with respect to the environment). A corresponding excess pressure can be created, for example, by heating an amount of the cosmetic preparation a) in a closed space to a temperature T₁. At a given temperature T₁, the liquid in the closed space has a saturation pressure p₁. For example, if the closed space is opened by employing a valve to a relaxation space, which is not under pressure and is at the pressure p₀<p₁, then the pressure in the previously closed space drops and the cosmetic preparation a) evaporates as part of the propagation of the new pressure level and/or the solvent or portions of the solvent contained in the cosmetic preparation evaporates. The resulting vapor or spray mist may be used for application of specific cosmetic preparations.

If the cosmetic preparation a) is heated in a closed space, i.e., starting from standard conditions (T₀=25° C., p₀=1.000 bar), then the further result is an increased pressure of the cosmetic preparation a) in addition to the increased temperature. This increased pressure can be relieved in a relaxation space to a pressure p₀, for example, the pressure of ambient air (p₀=1.000 bar), so that the cosmetic preparation a) can be evaporated at least partially.

The cosmetic preparation a) can be depressurized directly into the space in which it was previously heated. Alternatively, however, the heated cosmetic preparation a), which is under an excess pressure, may also be transported after heating to a second space, in which the pressure is then released.

In other words, flash vaporization is a process in which the cosmetic preparation a) in a closed container is heated by employing a heating apparatus to temperatures above ambient temperature, such that a pressure higher than ambient pressure develops in the container, and the heated cosmetic preparation a), which is then under an elevated pressure, is released out of the container and into the environment.

The device for flash vaporization is thus a device comprising a container and a heating apparatus and is designed so that a cosmetic preparation a) can be heated in the closed container by employing the heating apparatus to temperature above ambient temperature in such a manner that a pressure above ambient pressure occurs in the container, and the heated cosmetic preparation a), which is under an elevated pressure, can be released from the container into the environment.

Simultaneously with or after the pressure release, the cosmetic preparation a) can be sent to a nozzle such that the properties of the vapor and/or spray mist created by the flash vaporization, for example, in particular the droplet size or the droplet density but also the spray width and the shape of the spray cone, can be influenced. The use of nozzles, preferably atomizer nozzles, is therefore preferred. The specific type of nozzle or the specific shape of the nozzle is defined in a targeted manner as a function of the respective spray mist properties.

In summary a preferred device for flash vaporization comprises:

b1) a container b1), which is to be opened and closed by employing a valve and which defines the closed interior space in which the cosmetic preparation can be accommodated,
b2) a heating apparatus b2), which makes it possible to heat a cosmetic preparation that is in the container b1).

The use of an additional nozzle b3) which permits atomization of the cosmetic preparation a) escaping from the container is especially preferred. As an alternative to a valve, a closing element which has a comparable effect and can close or release a respective opening in the container through corresponding change in position may be used.

A preferred subject matter of the present disclosure is a cosmetic product, comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) wherein the device for flash vaporization comprises a container b1) and a heating apparatus b2) and is designed, so that
the cosmetic preparation a) can be accommodated in the interior space of the container b1),
the interior space of the container b1), which is filled at least partially with the cosmetic preparation a) can be sealed,
   the cosmetic preparation a) in the closed interior space of the container b1) may be heated by employing the heating apparatus b2), with an increase in the pressure.

An especially preferred subject matter of the present disclosure is a cosmetic product, comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) comprising
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a).

In other words, an especially preferred subject matter of the present disclosure is a cosmetic product, comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) wherein the device for flash vaporization comprises a container b1) and a heating apparatus b2) and is designed so that
the cosmetic preparation a) can be accommodated in the interior space of the container b1),
the interior space of the container b1), which is filled at least partially with the cosmetic preparation a), can be sealed,
the cosmetic preparation a) in the closed interior space of the container b1) may be heated by employing the heating apparatus b2), with an increase in the pressure,
the heated cosmetic preparation a) can be released from the interior space of the container b1) into the environment with a drop in pressure.

The container b1), in which the cosmetic preparation is heated, is designed to allow sealing of this container completely with respect to the environment during the heating of the cosmetic preparation a) and then, after heating, to open the container to allow flash vaporization of the cosmetic preparation a). This may be ensured, for example, by a part for regulating the flow, in particular a valve.

The container b1) in which the cosmetic preparation is heated is preferably in contact with another container, out of which the amount of cosmetic preparation provided for flash vaporization, is transferred to container b1) before heating. The access between this storage and container b1) is to be opened and closed by employing a corresponding device, for example, a valve. This additional container is preferably designed in the form of a storage container, i.e., it preferably comprises a multiple of the amount of cosmetic preparation needed for a vaporization process, preferably being many times larger, for example, more than about 10 times larger, preferably more than about 50 times larger than the amount required for a vaporization process. In other words, the additional container/storage container preferably comprises a multiple of the volume of the container b1), for example, more than about 10 times the volume, preferably more than about 20 times and in particular about 50 times the volume of container b1).

Another particularly preferred subject matter of the present disclosure is therefore a cosmetic product, comprising

a) a cosmetic preparation containing, based on its total weight:
a1) at least about 88% by weight polar solvent;
a2) at least about 0.1% by weight film-forming polymer;
wherein the amount by weight of components a) and b) relative to the total weight of the cosmetic preparation a) is more than about 92%;
b) a device for flash vaporization of the cosmetic preparation a), comprising:
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a);
c) a storage container for the cosmetic preparation a) out of which the cosmetic preparation a) can go to the container b1), wherein

the access between the storage container and container b1) has a part for flow regulation such that the flow rate of the cosmetic preparation a) out of the storage container and into the container b1) can be interrupted;

the storage container comprises at least 10 times the volume, preferably at least 20 times the volume and in particular at least five time the volume of container b1).

The storage container is not a pressurized container and the cosmetic preparation in the storage container is not under pressure. In other words, the pressure in the interior of the storage container corresponds to ambient pressure (also referred to atmospheric pressure or air pressure). Thus, corresponding cosmetic products, for example, do not include a propellant. The cosmetic product also does not have a pump device suitable for releasing or spraying the cosmetic preparation into the environment without the action of the device for flash vaporization.

A most especially preferred subject matter of the present disclosure is therefore a cosmetic product comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) comprising
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a);
c) a storage container for the cosmetic preparation a) out of which the cosmetic preparation a) can go to the container b1), wherein

the access between the storage container and container b1) has a part for flow regulation such that the flow rate of the cosmetic preparation a) out of the storage container and into the container b1) can be interrupted;

the storage container has a volume at least about 10 times greater, preferably at least about 50 times greater than that of b1);

the pressure in the interior of the storage container corresponding to the ambient pressure.

A most especially preferred subject matter of the present disclosure is therefore a cosmetic product, comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) comprising
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a);
c) a storage container for the cosmetic preparation a) out of which the cosmetic preparation a) can go to the container b1), wherein

the access between the storage container and container b1) has a part for flow regulation such that the flow rate of the cosmetic preparation a) out of the storage container and into the container b1) can be interrupted;

the storage container has a volume at least 10 times greater, preferably at least 50 times greater than that of b1)

the pressure in the interior of the storage container corresponds to the ambient pressure and the cosmetic product does not include a propellant.

In addition, cosmetic products are preferred that comprise

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) comprising
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a);
c) a storage container for the cosmetic preparation a) out of which the cosmetic preparation a) can go to the container b1), wherein

the access between the storage container and container b1) has a part for flow regulation such that the flow rate of the cosmetic preparation a) out of the storage container and into the container b1) can be interrupted;

the storage container has a volume at least about 10 times greater, preferably at least about 50 times greater than that of b1);

the pressure in the interior of the storage container corresponds to ambient pressure, wherein the cosmetic product does not have a pump device suitable for releasing or spraying the cosmetic preparation a) without the action of the device for flash vaporization.

In summary, a particularly preferred subject matter of the present disclosure is therefore a cosmetic product comprising

a) a cosmetic preparation containing, based on its total weight:
a1) at least about 88% by weight polar solvent;
a2) at least about 0.1% by weight film-forming polymer;

wherein the amount by weight of components a) and b) relative to the total weight of the cosmetic preparation a) is more than about 92%;

b) a device for flash vaporization of the cosmetic preparation a) comprising
b1) a container b1) that is to be opened and closed by employing a valve,
b2) a heating apparatus, which makes it possible to heat a cosmetic preparation in the container b1),
b3) a nozzle b3) which permits atomization of the cosmetic preparation a);
c) a storage container for the cosmetic preparation a) out of which the cosmetic preparation a) can go to the container b1), wherein

the access between the storage container and container b1) has a part for flow regulation such that the flow rate of the cosmetic preparation a) out of the storage container and into the container b1) can be interrupted;

the storage container has a volume at least about 10 times greater, preferably at least about 50 times greater than that of b1);

the pressure in the interior of the storage container corresponds to the ambient pressure and the cosmetic product does not include a propellant,

wherein the cosmetic product does not have a pump device suitable for releasing or spraying the cosmetic preparation a) without the action of the device for flash vaporization.

Additional care substances in particular can be mentioned as further suitable active ingredients or additives.

The agents as contemplated herein may additionally contain as a care substance at least one vitamin, a provitamins, a vitamin precursor and/or one of their derivatives. The vitamins, provitamins and vitamin precursors that are preferred as contemplated herein are those which are usually assigned to the groups A, B, C, E, F and H.

Additional care substances include panthenol, caffeine, nicotinamide and sorbitol.

The agents as contemplated herein may also contain as a care substance at least one plant extract but also mono- and/or oligosaccharides and/or lipids.

In addition to the two ingredients a1) and a2) described above, the cosmetic preparations a) as contemplated herein may contain additional active ingredients or additives, wherein in particular those active ingredients or additives which improve the producability, applicability and/or cosmetic effect of the cosmetic preparations as contemplated herein are preferred.

A first example of a preferred active ingredient and additive includes the cationic surfactants a3). Preferred cationic surfactants a3) are selected from quaternary ammonium compounds, esterquats and amidoamines. The cationic surfactants are contained in the cosmetic preparation a), based on its total weight, in amounts of about 0.01 to about 4.0% by weight, preferably about 0.05 to about 2.0% by weight and in particular 0.1 to 1.0% by weight. Cationic surfactants a3) from the group of quaternary ammonium compounds are especially preferred.

Preferred quaternary ammonium compounds include ammonium halides, in particular chlorides and bromides such as alkyltrimethylammonium chlorides, dialkyldimethylammonium chlorides and trialkylmethylammonium chlorides, for example, cetyltrimethylammonium chloride, stearyltrimethylammonium chloride, di stearyldimethylammonium chloride, lauryldimethylammonium chloride, lauryldimethylbenzylammonium chloride and tricetylmethylammonium chloride as well as the imidazolium compounds known by the INCI designations quaternium-27 and quaternium-83. The long alkyl chains of the surfactants mentioned above preferably have 10 to 18 carbon atoms. Most especially preferred cosmetic preparations a) as contemplated herein are characterized in that they contain, based on their total weight, about 0.01 to about 4.0% by weight, preferably about 0.05 to about 2.0% by weight and in particular about 0.1 to about 1.0% by weight (C₁₂ to C₁₈) alkyltrimethylammonium salt(s).

A second particularly preferred ingredient of the cosmetic preparations a) includes the protein hydrolyzates a4). These substances, optionally derivatized, may be of natural or synthetic origin. The amount by weight of protein hydrolyzate a4) in the total weight of the cosmetic preparation a) preferably amounts to about 0.01 to about 2.0% by weight, especially about 0.02 to about 1.0% by weight and in particular about 0.05 to about 0.8% by weight.

The group of particularly preferred natural protein hydrolyzates, i.e., the hydrolyzates of natural proteins, can be divided into protein hydrolyzates of plant and animal origin.

Protein hydrolyzates of animal origin include, for example, elastin, collagen, keratin, silk and milk protein hydrolyzates which may also be in the form of salts. Such products are distributed under the brand names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda), for example.

Plant-based protein hydrolyzates include, for example, soy, almond, pea, potato and wheat protein hydrolyzates. Such products can be obtained under the brand names, for example, Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

The group of marine protein hydrolyzates comprises protein hydrolyzates of animal and plant origin. Marine protein hydrolyzates include, for example, collagen hydrolyzates of fish or algae, protein hydrolyzates of mussels and the pearl hydrolyzates. Examples of pearl hydrolyzates or pearl extracts as contemplated herein include the substance mixtures distributed under the brand names Pearl Protein Extract BG® or Crodarom® Pearl.

Protein hydrolyzates based on collagen, silk protein, keratin, milk protein, soy protein, almond protein, wheat protein or pearls are especially preferred. The use of hydrolyzates of keratin, silk protein and wheat protein is most especially preferred.

The composition of some especially preferred cosmetic preparations as contemplated herein can be taken from the following tables (amounts given in % by weight, based on the total weight of the cosmetic agent, unless otherwise indicated). With respect to additional preferred specific embodiments of these particularly preferred compositions, what was said above regarding the cosmetic preparations a) as contemplated herein also applies here mutatis mutandis.

| | Formula 1 | Formula 2 | Formula 3 | Formula 4 | Formula 5 |
|---|---|---|---|---|---|
| Polar solvent a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Optional additive | to 100 | to 100 | to 100 | to 100 | to 100 |

| | Formula 6 | Formula 7 | Formula 8 | Formula 9 | Formula 10 |
|---|---|---|---|---|---|
| Water a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Optional additive | to 100 | to 100 | to 100 | to 100 | to 100 |

| | Formula 11 | Formula 12 | Formula 13 | Formula 14 | Formula 15 |
|---|---|---|---|---|---|
| Water and ethanol a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Optional additive | to 100 | to 100 | to 100 | to 100 | to 100 |

| | Formula 16 | Formula 17 | Formula 18 | Formula 19 | Formula 20 |
|---|---|---|---|---|---|
| Polar solvent a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Optional additive | to 100 | to 100 | to 100 | to 100 | to 100 |

| | Formula 21 | Formula 22 | Formula 23 | Formula 24 | Formula 25 |
|---|---|---|---|---|---|
| Water a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Optional additive | to 100 | to 100 | to 100 | to 100 | to 100 |

| | Formula 26 | Formula 27 | Formula 28 | Formula 29 | Formula 30 |
|---|---|---|---|---|---|
| Water and ethanol a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Optional additive | to 100 | to 100 | to 100 | to 100 | to 100 |

| | Formula 31 | Formula 32 | Formula 33 | Formula 34 | Formula 35 |
|---|---|---|---|---|---|
| Polar solvent a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Optional additive | to 100 | to 100 | to 100 | to 100 | to 100 |

| | Formula 36 | Formula 37 | Formula 38 | Formula 39 | Formula 40 |
|---|---|---|---|---|---|
| Water a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Optional additive | to 100 | to 100 | to 100 | to 100 | to 100 |

| | Formula 41 | Formula 42 | Formula 43 | Formula 44 | Formula 45 |
|---|---|---|---|---|---|
| Water and ethanol a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Optional additive | to 100 | to 100 | to 100 | to 100 | to 100 |

| | Formula 46 | Formula 47 | Formula 48 | Formula 49 | Formula 50 |
|---|---|---|---|---|---|
| Polar solvent a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 51 | Formula 52 | Formula 53 | Formula 54 | Formula 55 |
|---|---|---|---|---|---|
| Water a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 56 | Formula 57 | Formula 58 | Formula 59 | Formula 60 |
|---|---|---|---|---|---|
| Water and ethanol a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 61 | Formula 62 | Formula 63 | Formula 64 | Formula 65 |
|---|---|---|---|---|---|
| Polar solvent a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 66 | Formula 67 | Formula 68 | Formula 69 | Formula 70 |
|---|---|---|---|---|---|
| Water a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 71 | Formula 72 | Formula 73 | Formula 74 | Formula 75 |
|---|---|---|---|---|---|
| Water and ethanol a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 76 | Formula 77 | Formula 78 | Formula 79 | Formula 80 |
|---|---|---|---|---|---|
| Polar solvent a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 81 | Formula 82 | Formula 83 | Formula 84 | Formula 85 |
|---|---|---|---|---|---|
| Water a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 86 | Formula 87 | Formula 88 | Formula 89 | Formula 90 |
|---|---|---|---|---|---|
| Water and ethanol a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Cationic surfactant a3) | 0.01 to 4.0 | 0.05 to 2.0 | 0.1 to 1.0 | 0.15 | 0.15 |
| Optional additive | to 100 | to 100 | to 100 | to 100 | to 100 |

| | Formula 91 | Formula 92 | Formula 93 | Formula 94 | Formula 95 |
|---|---|---|---|---|---|
| Polar solvent a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 96 | Formula 97 | Formula 98 | Formula 99 | Formula 100 |
|---|---|---|---|---|---|
| Water a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 101 | Formula 102 | Formula 103 | Formula 104 | Formula 105 |
|---|---|---|---|---|---|
| Water and ethanol a1) | 88 to 99 | 90 to 98 | 92 to 97 | 93 | 98 |
| Film-forming polymer a2) | 0.1 to 10 | 0.2 to 8.0 | 0.4 to 5.0 | 2.5 | 0.5 |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 106 | Formula 107 | Formula 108 | Formula 109 | Formula 200 |
|---|---|---|---|---|---|
| Polar solvent a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 111 | Formula 112 | Formula 113 | Formula 114 | Formula 115 |
|---|---|---|---|---|---|
| Water a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 116 | Formula 117 | Formula 118 | Formula 119 | Formula 120 |
|---|---|---|---|---|---|
| Water and ethanol a1) | 88 to 99 | 90 to 98 | 92 to 97 | 93 | 98 |
| Vinylpyrrolidone/vinyl | 0.1 to 10 | 0.2 to 8.0 | 0.4 to 5.0 | 2.5 | 0.5 |
| acetate copolymer | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 121 | Formula 122 | Formula 123 | Formula 124 | Formula 25 |
|---|---|---|---|---|---|
| Polar solvent a1) | 88 to 99 | 90 to 98 | 92 to 97 | 93 | 98 |
| Polyurethane/preferably | 0.1 to 10 | 0.2 to 8.0 | 0.4 to 5.0 | 2.5 | 0.5 |
| Polyurethane-48 | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 126 | Formula 127 | Formula 128 | Formula 129 | Formula 130 |
|---|---|---|---|---|---|
| Water a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethane-48 | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

| | Formula 131 | Formula 132 | Formula 133 | Formula 134 | Formula 135 |
|---|---|---|---|---|---|
| Water and ethanol a1) | 88 to 99 | 90 to 98 | 92 to 97 | 93 | 98 |
| Polyurethane, preferably | 0.1 to 10 | 0.2 to 8.0 | 0.4 to 5.0 | 2.5 | 0.5 |
| Polyurethan-48 | | | | | |
| Protein hydrolyzate a4) | 0.01 to 2.0 | 0.02 to 1.0 | 0.05 to 0.8 | 0.2 | 0.1 |
| Optional additive | to 100 | to 100 | to 100 | to 100 | to 100 |

Most especially preferred cosmetic preparations contain only small amounts of other active ingredients and additives in addition to the ingredients a1) to a) already described above. Cosmetic preparations that are characterized in that the amount by weight of the ingredients a1), a2) and, if present, a3) and/or a4) relative to the total weight of the cosmetic preparation amounts to at least about 93% by weight, preferably at least about 95% by weight and in particular at least about 97% by weight, are especially preferred because of their ease of production and good cosmetic effect. Most especially preferred cosmetic preparations consist of at least about 94% by weight, preferably at least about 96% by weight, of ingredients a1) and a2), based on their total weight.

As stated in the introduction, the cosmetic preparations a) as contemplated herein are especially suitable for application by employing a device for flash vaporization. Another subject matter of the present patent application is therefore the use of a cosmetic preparation a) containing, based on its total weight

a1) at least about 88% by weight polar solvent;
a2) at least about 0.1% by weight film-forming polymer,
wherein the amount by weight of components a1) and a2) in the total weight of the cosmetic preparation a) amounts to more than about 92%, as the process material in a device for flash vaporization.

The subject matter of the present disclosure is also the use of a product as contemplated herein for treating keratin-containing fibers, in particular human hair, with a cosmetic preparation a) and/or for temporary shaping of keratin-containing fibers, in particular human hair.

One method for temporary shaping of keratin-containing fibers, in particular human hair in which the keratin-containing fibers are treated by employing a device for flash vaporization using a cosmetic preparation a) containing, based on its total weight:

a1) at least about 88% by weight polar solvent;
a2) at least about 0.1% by weight film-forming polymer,
wherein the amount by weight of components a1) and a2) in the total weight of the cosmetic preparation a) amounts to more than about 92%, is another subject matter of the present patent application. The cosmetic preparation a) is preferably converted to a spray mist by employing the device for flash vaporization, wherein the spray mist is then used to treat the keratin-containing fibers.

The agents as contemplated herein have special advantages within the scope of heat-induced hair shaping. One method that is especially preferred as contemplated herein for temporary shaping of keratin-containing fibers, preferably human hair, in the course of which the keratin-containing fibers are subjected to a heat treatment at temperatures of about 50° C. to about 350° C., preferably about 80° C. to about 300° C., especially preferably about 120° C. to about 260° C. and in particular about 150° C. to about 220° C., is therefore characterized in that the keratin-containing fibers are treated with a cosmetic preparation a) before the heat treatment with the help of a cosmetic product as contemplated herein.

Another subject matter of the present patent application is the use of a cosmetic product as contemplated herein to achieve a heat protection effect as part of hair shaping processes in which the keratin-containing fibers are subjected to a heat treatment at temperatures of about 50° C. to about 350° C., preferably of about 80 to about 300° C., especially preferably of about 120 to about 260° C. and in particular of about 150 to about 220° C.

To achieve an adequate spray effect, the cosmetic preparation a) is preferably heated to temperature above the boiling point of the polar solvent or solvent mixture contained in the cosmetic preparation a).

If the polar solvent is water or a solvent mixture with a water content above about 50% by weight (based on the total weight of the solvent mixture), then the cosmetic preparation is preferably heated to temperature above about 100° C., preferably to temperatures of about 100° C. and about 240° C., especially preferably to temperatures of about 140° C. to about 160° C.

The excess pressure achieved by heating the cosmetic preparation a) preferably amounts to between about 1.1 and about 8 bar, especially between about 1.2 and about 4 bar in those case in which the polar solvent is water or a solvent mixture with a water content greater than about 50% by weight (based on the total weight of the solvent mixture).

A preferred subject matter of the present patent application is a method for changing the color of keratin-containing fibers, in particular human hair, in which the keratin-containing fibers are treated by employing a device for flash vaporization with a cosmetic preparation a) containing, based on their total weight

a1) at least about 88% by weight polar solvent;
a2) at least about 0.1% by weight film-forming polymer,
wherein the amount by weight of components a1) and a2) in the total weight of the cosmetic preparation a) amounts to more than about 92%, wherein

a partial quantity of the cosmetic preparation a) in the storage container is transferred from said storage container, in whose interior a pressure corresponding to ambient pressure prevails, to a container b1);

subsequently, the access between the storage container and container b1) is interrupted by a part for flow regulation, such that the flow of the cosmetic preparation a) from the storage container into the container b1) can be interrupted;

subsequently, the cosmetic preparation a) present in the container b1) that is sealed with respect to the environment is heated by employing a heating apparatus, so that the pressure in the interior of the container b1) increases to levels above ambient pressure, preferably to levels between about 1.1 and about 8 bar, in particular to levels between about 1.2 and about 4 bar;

subsequently, the container b1), which is at a pressure above ambient pressure, is opened in such a way that it depressurizes the discharge of at least a partial quantity, preferably at least about 50% by weight, especially at least about 80% by weight and in particular at least about 90% by weight of the cosmetic preparation in the container b1) out of container b1) and into the environment with a reduction in the pressure prevailing in container b1) at the time of opening of the container.

Depressurization of the cosmetic preparation a) into the environment preferably takes place with the formation of a spray mist of the cosmetic preparation a).

The cosmetic preparation a) depressurized out of container b1 is preferably applied to keratinic fibers, in particular human hair.

Methods in the course of which the cosmetic preparation depressurized out of the container b1) is sent through a nozzle before treatment of the keratinic fibers are especially preferred.

With regard to additional preferred embodiments of the applications and the processes as contemplated herein, what was said above regarding the cosmetic preparations a) as contemplated herein and the device for flash pressurization b) also applies here mutatis mutandis.

In summary, the agents, applications and processes as contemplated herein and some of their preferred embodiments are characterized by the following points:

1. A cosmetic product, comprising
a) a cosmetic preparation containing, based on its total weight
a1) at least about 88% by weight polar solvent;
a2) at least about 0.1% by weight film-forming polymer;
b) a device for flash vaporization of the cosmetic preparation a),

wherein the amount by weight of components a1) and a2) in the total weight of the cosmetic preparation a) amounts to more than about 92%.

2. The cosmetic product according to point 1, characterized in that the device comprise a container (b1) and a heating apparatus b2) for flash vaporization and is designed so that

the cosmetic preparation a) can be accommodated in the interior space of the container b1),

the interior space of the container b1) that can be filled at least partially with the cosmetic preparation a) can be sealed,

the cosmetic preparation a) can be heated in the closed interior space of the container b1) by employing the heating apparatus b2) with an increase in pressure,

the heated cosmetic preparation a) can be released out of the interior space of the container b1) and into the environment with a reduction in pressure.

3. The cosmetic product according to any one of the preceding points, characterized in that the amount by weight of the polar solvent a1) in the total weight of the cosmetic preparation a) amounts to about 88% to about 98% by weight, preferably about 90% to about 98% by weight and in particular about 92% to about 97% by weight.
4. The cosmetic product according to any one of the preceding points, characterized in that the polar solvent a1) has a boiling point (20° C., 1013 mbar) between about 50° C. and about 110° C., preferably between about 70° C. and about 105° C.
5. The cosmetic product according to any one of the preceding points, characterized in that the polar solvent a1) is selected from the group of ethanol and water.
6. The cosmetic product according to any one of the preceding points, characterized in that the amount by weight of water and ethanol in the total weight of the polar solvent a1) preferably amount to at least about 80% by weight, especially at least about 85% by weight, especially preferably at least about 90% by weight and in particular at least about 95% by weight.
7. The cosmetic product according to any one of the preceding points, characterized in that the amount by weight of water in the total weight of the polar solvent a1) amounts to more than about 80% by weight, preferably more than about 85% by weight and in particular more than about 90% by weight.
8. The cosmetic product according to any one of the preceding points, characterized in that the polar solvent a1) comprises water and ethanol and the weight ratio of water to ethanol amounts to about 50:1 to about 1:1, preferably about 40:1 to about 5:1 and in particular about 25:1 to about 10:1.
9. The cosmetic product according to any one of the preceding points, characterized in that the amount by weight of the film-forming polymer a2) in the total weight of the cosmetic preparation a) amounts to about 0.1 to about 10% by weight, preferably about 0.2 to about 8.0% by weight and in particular about 0.4 to about 5.0% by weight.
10. The cosmetic product according to any one of the preceding points, characterized in that the film-forming polymer a2) is selected from the group of nonionic polymers, preferably from the group of polyvinylpyrrolidones and vinylpyrrolidone/vinyl acetate copolymers, in particular vinylpyrrolidone/vinyl acetate copolymers.
11. The cosmetic product according to any one of the preceding points, characterized in that the film-forming polymer a2) is selected from the group of polyurethanes, preferably from the group of anionic polyurethane polymers comprising at least one structural unit of formula (I) and at least one structural unit of formula (II) and at least one structural unit of formula (III) and at least one structural unit of formula (IV) and at least one structural unit of formula (V):

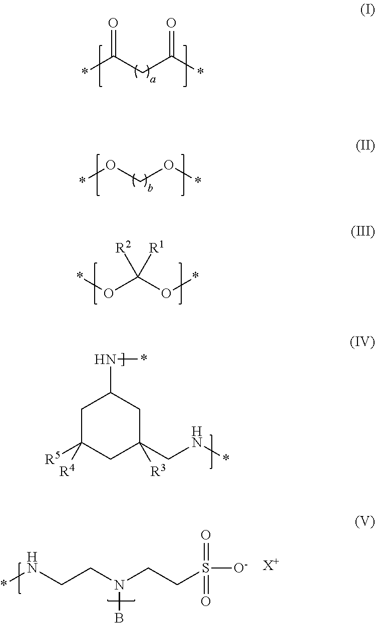

where

a and b independently of one another stand for integers from 1 to 20,

R¹, R², R³, R⁴ and R⁵ each independently of one another stands for a hydrogen atom or a C₁-C₄ alkyl group,

B stands for a hydrogen atom or a direct bond to another structural unit,

X⁺ stands for a physiologically tolerable cation.

12. The cosmetic product according to any one of the preceding points, characterized in that the cosmetic preparation a) contains, based on its total weight about 0.01 to about 4.0% by weight, preferably about 0.05 to about 2.0% by weight and in particular about 0.1 to about 1.0% by weight of a cationic surfactant a3).
13. The cosmetic product according to point 12, characterized in that the cationic surfactant a3) is selected from the groups of quaternary ammonium compounds, esterquats and amidoamines, preferably from the group of quaternary ammonium compounds, in particular from the group of C₁₂ to C₁₈ alkyltrimethylammonium salts.
14. The cosmetic product according to any one of the preceding points, characterized in that the cosmetic preparation a) contains, based on its total weight about 0.01 to about 2.0% by weight, preferably about 0.02 to about 1.0% by weight and in particular about 0.05 to about 0.8% by weight of at least one protein hydrolyzate a4).
15. The cosmetic product according to any one of the preceding points, characterized in that the protein hydrolyzate is selected from the group of hydrolyzates of keratin, silk protein and wheat protein.
16. The cosmetic product according to any one of the preceding points, characterized in that the cosmetic preparation a) comprises, based on its total weight at least 94% by weight, preferably at least about 96% by weight of the components a1) and a2).
17. The cosmetic product according to any one of the preceding points, characterized in that the cosmetic preparation a) comprises, based on the total weight at least about 93% by weight, preferably at least about 95% by weight and in particular at least about 97% by weight of the components a1), a2), a3) and a4).
18. Use of a cosmetic preparation a) containing, based on its total weight
a) a cosmetic preparation containing, based on its total weight
a1) at least about 88% by weight polar solvent;
a2) at least about 0.1% by weight film-forming polymer,
wherein the amount by weight of components a1) and a2) in the total weight of the cosmetic preparation a) amounts to more than about 92%, as the process material in a device for flash vaporization.
19. Use of a product according to any one of points 1 to 17 for treating keratin-containing fibers, in particular human hair, with a cosmetic preparation a).
20. Use of a product according to any one of points 1 to 17 for temporary shaping of keratin-containing fibers, in particular human hair.
21. A method for temporary shaping of keratin-containing fibers, in particular human hair, in which the keratin-containing fibers are treated by employing a device for flash vaporization using a cosmetic preparation a) containing, based on its total weight
a1) at least about 88% by weight polar solvent,
a2) at least about 0.1% by weight film-forming polymer,

wherein the amount by weight of components a1) and a2) in the total weight of the cosmetic preparation a) amounts to more than about 92%.

22. The method according to point 21, characterized in that

a partial amount of the cosmetic preparation a) in a storage container is transferred to a container b1) from a storage container in the interior of which a pressure corresponding to ambient pressure prevails;

subsequently, the access between the storage container and container b1) is interrupted by a component for flow control such that the flow of the cosmetic preparation a) out of the storage container and into container b1) can be interrupted;

subsequently, the cosmetic preparation a) in the container b1) which is sealed with respect to the environment is heated by employing a heating apparatus so that the pressure in the interior of the container b1) rises to levels above ambient pressure, preferably to levels between about 1.1 and about 8 bar, in particular to levels between about 1.2 and about 4 bar;

subsequently, the container b1), which is under a pressure above ambient pressure is opened in such a way that it depressurizes the escape of at least a partial amount, preferably at least about 50% by weight, especially at least about 80% by weight and in particular at least about 90% by weight of the cosmetic preparation in the container b1) out of container b1) and into the environment with a drop in the pressure prevailing in container b1) at the time of opening of the container.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product, comprising:
a) a cosmetic preparation comprising, based on its total weight,
a1) at least about 88% by weight polar solvent; and
a2) at least about 0.1% by weight film-forming polymer; and
b) a device for flash vaporization of the cosmetic preparation a);
wherein the amount by weight of components a1) and a2) in the total weight of the cosmetic preparation a) amounts to more than about 92%.

2. The cosmetic product according to claim 1, wherein the device comprises a container (b1) and a heating apparatus b2) for flash vaporization and is designed so that;
the cosmetic preparation a) can be accommodated in the interior space of the container b1),
the interior space of the container b1) that can be filled at least partially with the cosmetic preparation a) can be sealed,
the cosmetic preparation a) can be heated in the closed interior space of the container b1) by employing the heating apparatus b2) with an increase in pressure, and
the heated cosmetic preparation a) can be depressurized out of the interior space of the container b1) into the environment with a reduction in pressure.

3. The cosmetic product according to claim 1, wherein the amount by weight of the polar solvent a1) in the total weight of the cosmetic preparation a) amounts to about 88% to about 98% by weight.

4. The cosmetic product according to claim 1, wherein the polar solvent a1) is selected from the group of ethanol, water, and a combination thereof.

5. The cosmetic product according to claim 1, wherein the amount by weight of the film-forming polymer a2) in the total weight of the cosmetic preparation a) amounts to about 0.1 to about 10% by weight.

6. The cosmetic product according to claim 1, wherein the film-forming polymer a2) comprises nonionic polymers.

7. The cosmetic product according to claim 1, wherein the film-forming polymer a2) comprises polyurethanes.

8. A cosmetic preparation a) comprising, based on its total weight:
a1) at least about 88% by weight polar solvent; and
a2) at least about 0.1% by weight film-forming polymer;
wherein the amount by weight of components a1) and a2) in the total weight of the cosmetic preparation a) amounts to more than about 92%; and
wherein the cosmetic preparation is utilized as a material in a device for flash vaporization.

9. The cosmetic product according to claim 1, wherein the cosmetic product is utilized for treating keratin-containing fibers with the cosmetic preparation a).

10. The cosmetic product according to claim 1, wherein the cosmetic product is utilized for temporary shaping of keratin-containing fibers.

11. A method for temporary shaping of keratin-containing fibers, the method comprising:
treating the keratin-containing fibers by employing a device for flash vaporization using a cosmetic preparation a) comprising, based on its total weight:
a1) at least about 88% by weight polar solvent; and
a2) at least about 0.1% by weight film-forming polymer;
wherein the amount by weight of components a1) and a2) in the total weight of the cosmetic preparation a) amounts to more than about 92%.

12. The method according to claim 11, wherein:
a partial amount of the cosmetic preparation a) in a storage container is transferred to a container b1) from a storage container in whose interior a pressure prevails that corresponds to the ambient pressure;
subsequently, the access between the storage container and container b1) is interrupted by a component for flow control such that the flow of the cosmetic preparation a) out of the storage container and into container b1) can be interrupted;
subsequently, the cosmetic preparation a) in the container b1), which is sealed with respect to the environment is heated by employing a heating apparatus, so that the pressure in the interior of the container b1) rises to levels above ambient pressure; and
subsequently, the container b1) which is under a pressure above ambient pressure is opened such that it depressurizes the escape of at least a partial amount of the cosmetic preparation in the container b1) out of the container b1) and into the environment with a drop in the pressure prevailing in the container b1) at the time of opening of the container.

13. The cosmetic product according to claim 6, wherein the film-forming polymer a2) comprises polyvinylpyrrolidones, vinylpyrrolidone/vinyl acetate copolymers, or a combination thereof.

14. The cosmetic product according to claim 7, wherein the film-forming polymer a2) comprises anionic polyurethane polymers comprising at least one structural unit of formula (I) and at least one structural unit of formula (II) and at least one structural unit of formula (III) and at least one structural unit of formula (IV) and at least one structural unit of formula (V): 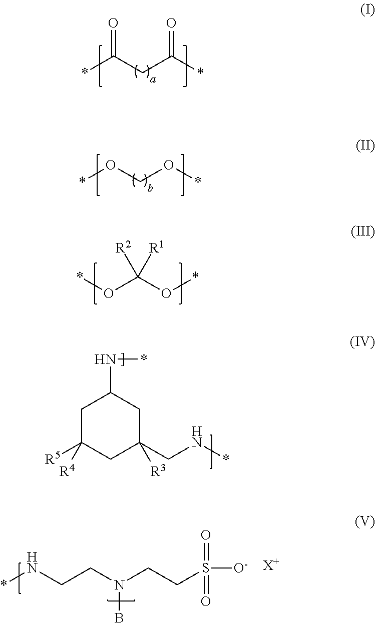
where
a and b, independently of one another, stand for integers from 1 to 20,
R¹, R², R³, R⁴ and R⁵, independently of one another, each stand for a hydrogen atom or a C₁-C₄ alkyl group,
B stands for a hydrogen atom or a direct bond to another structural unit, and
X⁺ stands for a physiologically tolerable cation.

15. The cosmetic product according to claim 1, wherein the cosmetic preparation a) comprises, based on its total weight about 0.01 to about 4.0% by weight of a cationic surfactant a3).

16. The cosmetic product according to claim 15, wherein the cationic surfactant a3) is selected from the group of quaternary ammonium compounds, esterquats, amidoamines, and combinations thereof.

17. The cosmetic product according to claim 16, wherein the cationic surfactant a3) is selected from the group of C₁₂ to C₁₈ alkyltrimethylammonium salts and combinations thereof.

18. The cosmetic product according to claim 1, wherein the cosmetic preparation a) comprises, based on its total weight about 0.01 to about 2.0% by weight of at least one protein hydrolyzate a4).

19. The cosmetic product according to claim 18, wherein the protein hydrolyzate a4) is selected from the group of hydrolyzates of keratin, silk protein, wheat protein, and combinations thereof.

20. The cosmetic product according to claim 1, wherein the cosmetic preparation a) comprises, based on its total weight at least about 94% by weight of the components a1) and a2).
